(19) United States
(12) Patent Application Publication
(10) Pub. No.: US 2006/0015170 A1
(43) Pub. Date: Jan. 19, 2006

(54) Contrast coated stent and method of fabrication

(76) Inventors: Ryan A. Jones, Santa Rosa, CA (US); John D, Kantor, Santa Rosa, CA (US); John Shanahan, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(21) Appl. No.: 10/893,548

(22) Filed: Jul. 16, 2004

(51) Int. Cl. A61F 2/84; A61F 2/90

(52) U.S. Cl. 623/1.12; 623/1.34; 623/1.42

(57) ABSTRACT

The invention provides a system for treating a vascular condition. The system includes a catheter and a stent disposed on the catheter. The system further includes a stent with at least an outer portion of the stent framework coated with a drug polymer solution and a contrast medium substantially covering at least an outer surface of the drug coating disposed on the outer surface of the stent framework. A method of manufacturing the contrast coated stent and a method of using the contrast coated stent is included.

### TECHNICAL FIELD

The technical field of this disclosure is medical implant devices, particularly, a contrast coated stent and methods of making and using the same.

### BACKGROUND OF THE INVENTION

Stents are generally cylindrical shaped devices that are radially expandable to hold open a segment of a blood vessel or other anatomical lumen after implantation into the body lumen. Stents have been developed with coatings to deliver drugs or other therapeutic agents.

Stents are used in conjunction with balloon catheters in a variety of medical therapeutic applications including intravascular angioplasty. For example, a balloon catheter device is inflated during PTCA (percutaneous transluminal coronary angioplasty) to dilate a stenotic blood vessel. The stenosis may be the result of a lesion such as a plaque or thrombus. After inflation, the pressurized balloon exerts a compressive force on the lesion thereby increasing the inner diameter of the affected vessel. The increased interior vessel diameter facilitates improved blood flow. Soon after the procedure, however, a significant proportion of treated vessels re-narrow.

To prevent restenosis, short flexible cylinders, or stents, constructed of metal or various polymers are implanted within the vessel to maintain lumen size. The stent acts as a scaffold to support the lumen in an open position. Various configurations of stents include a cylindrical tube defined by a mesh, interconnected stents or like segments. Some exemplary stents are disclosed in U.S. Pat. No. 5,292,331 to Boneau, U.S. Pat. No. 6,090,127 to Globerman, U.S. Pat. No. 5,133,732 to Wiktor, U.S. Pat. No. 4,739,762 to Palmaz and U.S. Pat. No. 5,421,955 to Lau. Balloon-expandable stents are mounted on a collapsed balloon at a diameter smaller than when the stents are deployed. Stents can also be self-expanding, growing to a final diameter when deployed without mechanical assistance from a balloon or like device.

Stents have been used with coatings to deliver drug or other therapy to the patient at the site of the stent, such as the interior wall of an artery or vessel. The coating is typically applied to the stent by dipping or spraying the stent with a liquid containing the drug or therapeutic agent dispersed in a polymer/solvent mixture. The liquid coating then dries to a solid uniform coating. Combinations of dipping and spraying can also be used. The dried coating forms a uniform radial layer over the stent elements. Some stents may have several layers of drug and polymer coatings.

The drug/polymer coated stent is then placed on a catheter for delivery to the treatment site. The coated stent may be placed over a balloon or, if self-expanding, it may be placed within a retractable sheath. Once the coated stent has been positioned on the delivery catheter, the stent catheter assembly is typically placed within a protective sheath for shipping and handling.

Damage to the coating may occur, however, during the handling and deployment of drug-coated stents. The coating may be rubbed off when the stent catheter assembly is placed in the protective sheath and/or when the coated stent is advanced through the delivery catheter during placement. Damage to the coating may also occur while the stent is advancing through the patients vascular system on the way to the treatment site. Still other damage to the coating may occur when the stent is expanded by the balloon. Damage to the drug coat during each of these situations may result in delivery of a less-than-effective dose of the drug at the treatment site.

Visualizing the stent during the advancement of the stent through the patient's vascular system also poses problems for the doctor performing the procedure. Stents are often composed of material that is not easily visualized by the doctor as it travels through the body while other stents are so small they are hard to visualize.

It would be desirable, therefore, to have a drug-coated stent and method of making the same that would overcome the above disadvantages.

### SUMMARY OF THE INVENTION

One aspect of the present invention provides a system for treating a vascular condition. The system includes a catheter and a stent disposed on the catheter. The system further includes a stent having a stent framework, a drug coating disposed on the stent framework, and a contrast medium substantially covering at least an outer surface of the drug coating disposed on an outer surface of the stent framework.

Another aspect of the present invention provides a method of treating a vascular condition using a contrast coated stent. The method includes the steps of delivering a drug coated stent with contrast coating to a target region of a vessel via a catheter, dissolving the coating while the stent is delivered to the target region and deploying the stent at the target region.

Another aspect of the present invention provides a method of protecting a drug coated stent. The method includes the steps of applying a contrast medium solution to at least an outer surface area of a drug-coated stent and drying the applied contrast medium solution to form a contrast coating.

The foregoing and other features and advantages of the invention will become further apparent from the following detailed description of the presently preferred embodiments, read in conjunction with the accompanying drawings. The detailed description and drawings are merely illustrative of the invention, rather than limiting the scope of the invention being defined by the appended claims and equivalents thereof.

### BRIEF DESCRIPTION OF THE DRAWINGS

FIG. 1 is an illustration of a system for treating a vascular condition including a contrast-drug coated stent coupled to a catheter, in accordance with one embodiment of the current invention;

FIG. 2 is a cross-sectional view of a contrast-drug coated stent, in accordance with one embodiment of the current invention;

FIG. 3 is a cross-sectional view of a contrast-drug coated stent, in accordance with another embodiment of the current invention;

FIG. 4 is a flow diagram of a method of manufacturing a drug-polymer coated stent, in accordance with one embodiment of the current invention; and

FIG. 5 is a flow diagram of a method of treating a vascular condition including a contrast-drug coated stent, in accordance with one embodiment of the current invention.

### DETAILED DESCRIPTION OF THE PRESENTLY PREFERRED EMBODIMENT

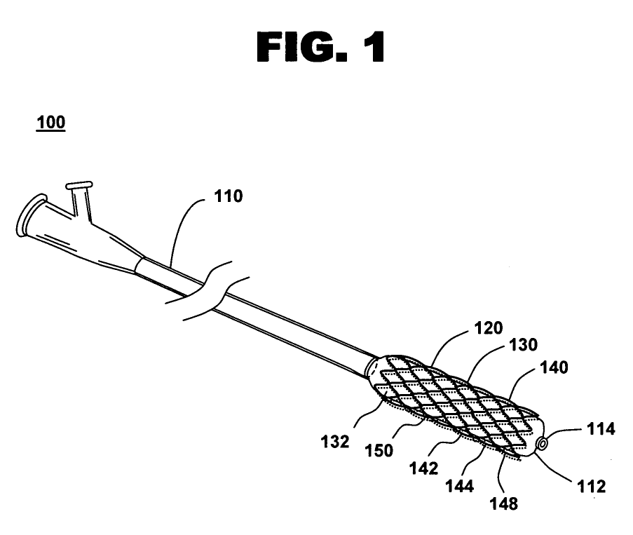

FIG. 1 illustrates a system for treating a vascular condition, comprising a contrast-drug polymer coated stent coupled to a catheter, in accordance with one embodiment of the present invention at 100. Vascular condition treatment system 100 includes a contrast-drug polymer coated stent 120 coupled to a delivery catheter 110. Coated stent 120 includes a stent framework 130, a drug-polymer coating 140 disposed on at least a portion of stent framework 130 and a contrast coat disposed on drug-polymer coating 140.

The stent 120 is conventional to stents generally and can be made of a wide variety of medical implantable materials, such as stainless steel (particularly 316-L or 316LS stainless steel), MP35 alloy, nitinol, tantalum, ceramic, nickel, titanium, aluminum, polymeric materials, tantalum, MP35N, titanium ASTM F63-83 Grade 1, niobium, high carat gold K 19-22, and combinations thereof. The stent 120 can be formed through various methods as well. The stent 120 can be welded, laser cut, molded, or consist of filaments or fibers which are wound or braided together in order to form a continuous structure. Generally tubular in shape with open ends, the latticework of stent framework 130 has a plurality of open apertures 132 between the struts, shaped to allow expansion of stent framework 130 from an initially contracted form when deployed. Depending on the material, the stent can be self-expanding, or be expanded by a balloon or some other device.

Drug polymer coating 140 includes a polymeric coating 142 positioned adjacent to stent framework 130 and at least one therapeutic agent 144 encased by or interdispersed within drug-polymer coating 140. In some cases, drug-polymer coating 140 includes a cap coating 148 disposed on drug-polymer coating 140. Drug-polymer coating 140 can provide time-released delivery of one or more therapeutic agents to surrounding tissue after coated stent 120 has been deployed within a vessel of the body.

Contrast coating 150 is disposed on the drug-polymer coating 140. Contrast coating 150 comprises a contrast medium as are well known in the art. Typical contrast mediums are high osmolar, injectable, ionic and non-ionic, biologically inert, and provide enhancement in CT, XRay, and Flouroscopy imaging procedures. Such contrast mediums may contain iothalamate meglumine, organically bound iodine, and other proprietary ingredients. Some contrast media may contain stabilizers such as edetate calcium disodium, and buffers such as sodium phosphate. Examples of contrast media agents are Conray®, Conray® 30, Conray® 43, Cysto Conray®, and Cysto Conray® II, Conray®-400, Omnipaque®, Renoghraphin®, and Hypaque®. Contrast coating 150 may be applied to the stent by dipping, spraying, brushing or combinations of dipping, spraying and brushing. Contrast coat 150 may be applied to the outside surface of the stent framework, the inside surface of the stent framework or both.

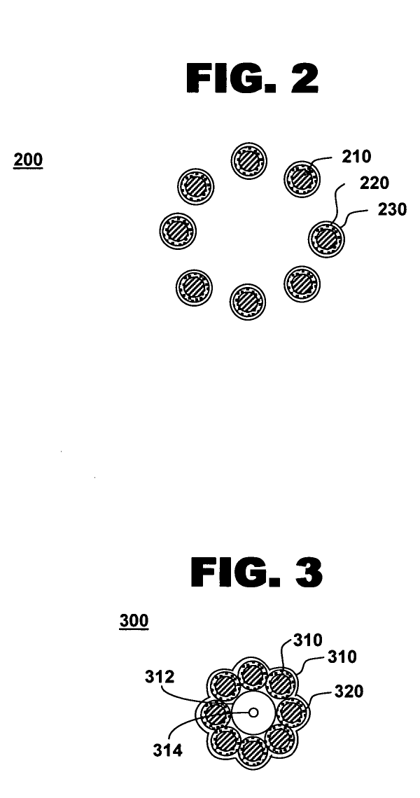

Contrast coat 150 may be applied to the stent before or after it is placed on the catheter. FIG. 2 illustrates a cross section of one embodiment of a contrast-coated stent 200 used in a system for treating a vascular condition 100 illustrated in FIG. 1. Contrast-coated stent 200 includes struts 210, drug-polymer coating 220 and contrast coating 230. FIG. 2 illustrates a stent 200 that is coated with the contrast medium before it is disposed on a delivery catheter or a balloon catheter. In this embodiment, the stent framework is in an expanded position so that the inner and outer surfaces of the drug-coated stent may be uniformly coated with contrast medium. The drug-polymer coating 220 and the contrast coating 230 may have been applied by dipping, spraying or a combination of both as is well known in the art. Contrast coated stent may have multiple layers of contrast medium disposed on the drug-polymer layer 220

FIG. 3 illustrates a cross section of another embodiment of a contrast-coated stent 300 used in a system for treating a vascular condition 100 illustrated in FIG. 1. Contrast-coated stent 300 includes struts 310, drug-polymer coating 320 and contrast coating 330. Contrast-coated stent 300 is disposed on balloon 312 in a manner well known in the art. Balloon 312 is disposed around guide wire lumen 314. Contrast coating 330 is applied to the stent/balloon assembly. Contrast coating 330 may be applied by dipping, spraying or a combination of both as is well known in the art.

Contrast coat 150 protects the drug-polymer layer from damage that may occur during shipping and handling. Specifically, it can protect the drug-polymer layer as the stent is prepared for shipping and insertion into the patient. Contrast coat 150 also protects the drug-polymer layer as the stent is advanced through the patients vascular system. Contrast coating 150 on an inner surface of a stent that is deployed on a balloon catheter helps protect the drug-polymer layer located adjacent the balloon as the balloon is inflated to expand the stent.

Contrast coat 150 dissolves as the coated stent advances to the treatment site. The rate of dissolution may be affected by the concentration of the contrast medium and the thickness of the contrast coat. The dissolving contrast coat aids in the visualization of the stent/catheter assembly as it traverses the patient's vascular system. The contrast medium may be applied to the stent in a concentration that would dissolve in a predetermined length of time. For example, the contrast coat may be applied so that it dissolves in about 20 to 60 seconds, i.e. the length of time necessary to place the stent at the treatment site. In another embodiment the contrast medium is coated onto the stent so that it dissolves in about 1 to 5 minutes, a sufficient amount of time to advance the stent to the treatment site, inflate a balloon and properly position the stent. Those with skill in the art will recognize that the contrast coat may be applied in a variety of concentrations that would aid in the proper positioning of the contrast-drug coated stent.

Insertion of contrast and drug-coated stent 120 into a vessel in the body helps treat, for example, heart disease, various cardiovascular ailments, and other vascular conditions. Catheter-deployed contrast coated stent 120 typically is used to treat one or more blockages, occlusions, stenoses, or diseased regions in the coronary artery, femoral artery, peripheral arteries, and other arteries in the body. Treatment of vascular conditions involves the prevention or correction of various ailments and deficiencies associated with the cardiovascular system, the cerebrovascular system, urinogenital systems, biliary conduits, abdominal passageways and other biological vessels within the body.

An exemplary drug-polymer coating 140 includes or encapsulates one or more therapeutic agents. Drug-polymer coating 140 may comprise one or more therapeutic agents 144 dispersed within or encased by drug-polymer coating 140 on contrast coated stent 120, which are eluted from contrast coated stent 120 with controlled time delivery after deployment of contrast coated stent 120 in the body. A therapeutic agent is capable of producing a beneficial effect against one or more conditions including coronary restenosis, cardiovascular restenosis, angiographic restenosis, arteriosclerosis, hyperplasia, and other diseases or conditions. For example, the therapeutic agent can be selected to inhibit or prevent vascular restenosis, a condition corresponding to a narrowing or constricting of the diameter of the bodily lumen where the stent is placed. Drug-polymer coating 140 may comprise, for example, an antirestenotic drug such as rapamycin, a rapamycin analogue, or a rapamycin derivative to prevent or reduce the recurrence or narrowing and blockage of the bodily vessel. Drug-polymer coating 140 may comprise an anti-cancer drug such as camptothecin or other topoisomerase inhibitors, an antisense agent, an antineoplastic agent, an antiproliferative agent, an antithrombogenic agent, an anticoagulant, an antiplatelet agent, an antibiotic, an anti-inflammatory agent, a steroid, a gene therapy agent, an organic drug, a pharmaceutical compound, a recombinant DNA product, a recombinant RNA product, a collagen, a collagenic derivative, a protein, a protein analog, a saccharide, a saccharide derivative, a bioactive agent, a pharmaceutical drug, a therapeutic substance, or a combination thereof.

The elution rates of the therapeutic agents and total drug eluted into the body and the tissue bed surrounding the stent framework are based on the thickness of drug-polymer coating 140; the constituency of drug-polymer coating 140; the nature, distribution and concentration of the therapeutic agents; the thickness and composition of any cap coat, and other factors. Drug-polymer coating 140 may include and elute multiple therapeutic agents to achieve the desired therapeutic effect. Drug-polymer coating 140 can be tailored to control the elution of one or more therapeutic agents that are transported through the coating primarily by diffusion processes. In some cases, a portion of the polymeric coating is absorbed into the body, releasing therapeutic agents embedded within or encased by the coating. In other cases, drug-polymer coating 140 erodes from coated stent 120 to release the therapeutic agents, the residual polymer being expelled by the body. Cap coating 148 can be selected to provide a diffusion barrier to the therapeutic agents and aid in the control of drug elution.

Incorporation of a drug or other therapeutic agents into drug-polymer coating 140 allows, for example, the rapid delivery of a pharmacologically active drug or bioactive agent within twenty-four hours following the deployment of a stent, with a slower, steady delivery of a second bioactive agent over the next three to six months. For example, the therapeutic agent may comprise an antirestenotic drug such as rapamycin, a rapamycin analogue, or a rapamycin derivative. A second therapeutic agent may comprise, for example, an anti-inflammatant such as dexamethasone. The therapeutic agent constituency in the drug-polymer coating may be, for example, between 0.1 percent and 90 percent of the drug-polymer coating by weight.

Catheter 110 of an exemplary embodiment of the present invention includes a balloon 112 that expands and deploys the stent within a vessel of the body. The balloon 112 may be any variety of balloons capable of expanding the stent 120. The balloon 110 may be manufactured from a material such as polyethylene, polyethylene terephthalate (PET), nylon, Pebax® polyether-block co-polyamide polymers, or the like. After positioning contrast-coated stent 120 within the vessel with the assistance of a guide wire traversing through a guidewire lumen 114 inside catheter 110, balloon 112 is inflated by pressurizing a fluid such as saline that fills a tube inside catheter 110 and balloon 112. Contrast-coated stent 120 is expanded until a desired diameter is reached, and then the fluid is depressurized or pumped out, separating balloon 112 from coated stent 120 and leaving drug-coated stent 120 deployed in the vessel. Alternatively, catheter 110 may include a sheath that retracts, allowing the expansion of a self-expanding version of contrast-coated stent 120.

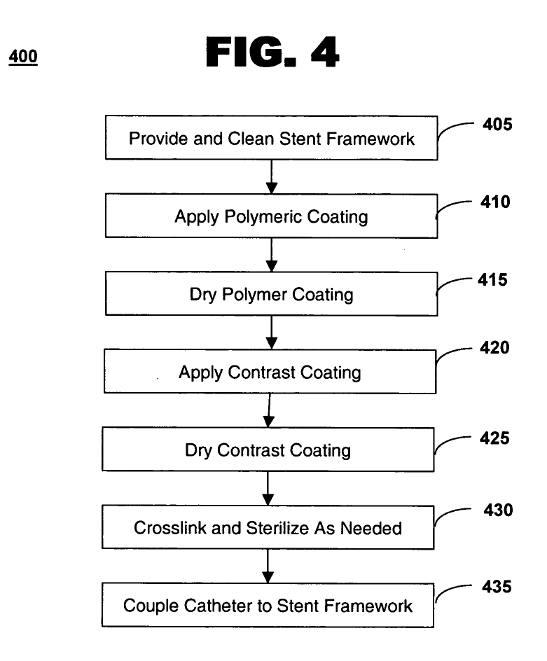

FIG. 4 shows a flow diagram for forming a contrast and drug-polymer coated stent, in accordance with one embodiment of the present invention at 400. Method 400 includes various steps to form a drug-polymer coating on a stent framework with a contrast coating.

A stent framework is provided and cleaned (Block 405). The stent framework may be cleaned, for example, by inserting the stent framework into various solvents, degreasers and cleansers to remove any debris, residues, or unwanted materials from the surface of the stent framework. The stent framework is dried, and generally inspected at this point in the process. Generally, a primer coating is not required, though a primer coating may be applied to the stent framework prior to application of the polymer or drug-polymer coating. The primer coating is dried to eliminate or remove any volatile components and then cured or crosslinked as needed. Excess liquid may be blown off prior to drying the primer coating, which may be done at room temperature or at elevated temperatures under dry nitrogen or other suitable environments including a vacuum environment.

A polymeric coating is applied onto at least a portion of the stent framework (Block 410). The polymeric coating may comprise, for example, a primer coating, a drug-polymer coating, a cap coating, or a combination thereof. The polymeric coating is applied using any suitable coating technique such as dipping, spraying, painting, or brushing. Exemplary applied polymeric coatings comprise polymers such as poly(vinyl alcohol), poly(ethylene-vinyl acetate), polyurethane, polycaprolactone, polyglycolide, poly(lactide-co-glycolide), poly(ethylene oxide), poly(vinyl pyrrolidone), silicone, an acrylic polymer, an acrylic and acrylonitrile copolymer, a latex polymer, a thermoplastic polymer, a thermoset polymer, a biostable polymer, a biodegradable polymer, a blended polymer, a copolymer, and combinations thereof. In one embodiment of the present invention, one or more therapeutic agents may be added to and dispersed within the polymeric coating before its application onto the stent framework.

The dipped, sprayed or brushed stent framework is then dried (Block 415). The coated stent framework may be dried, for example, by positioning the coated stent framework in air and evaporating any solvent from the applied polymeric coating. The polymeric coating is generally dried after application by evaporating the solvent at room temperature and under ambient conditions. A nitrogen environment or other controlled environment may also be used for drying. Alternatively, the polymeric coating can be dried by evaporating the majority of any solvent at room temperature, and then further drying the coating in a vacuum environment between, for example, a room temperature of about 25 degrees centigrade and 50 degrees centigrade or higher. Drying in a vacuum environment helps to extract any pockets of solvent buried within the polymeric coating and to provide the desired level of crosslinking in the polymer.

A contrast coat is applied onto the drug/polymer coated stent (Block 420). The contrast coat may comprise a contrast medium commonly used in x-ray technology. The contrast medium may be, for example, those discussed above or any other suitable contrast medium known in the art. The contrast medium is applied using any suitable coating technique such as dipping, spraying, painting, or brushing. The contrast medium may have a concentration of between about one percent and one hundred (100) percent. Those with skill in the art will recognize that the concentration of the contrast medium used may depend on such factors as, for example, the thickness of the coat to be applied, the length of time for deployment of the coated stent and the specific contrast medium utilized. The contrast medium may be diluted before application to the stent framework. For example, in one embodiment, the contrast medium is diluted with saline. The contrast medium may be diluted with any appropriate dilutant as is well known in the art.

In another embodiment, the contrast medium may be coated onto the stent with more than one application or coats. Several coats of contrast medium may be applied to achieve a specific thickness of contrast medium, a specific concentration or both. In one embodiment, each respective layer of contrast medium is dried before the next coat is applied.

The contrast-coated stent is then dried (Block 425). The contrast-coated stent framework may be dried, for example, by positioning the contrast coated stent framework in air and evaporating any solvent from the applied contrast medium coating. The contrast medium is generally dried after application by evaporating the solvent at room temperature and under ambient conditions. A nitrogen environment or other controlled environment may also be used for drying. Alternatively, the contrast medium can be dried by evaporating the majority of any solvent at room temperature, and then further drying the contrast coating in a vacuum environment between, for example, a room temperature of about 25 degrees centigrade and 50 degrees centigrade or higher.

The contrast-coated stent may be crosslinked and sterilized as needed (Block 430). Cross-linking may be done by providing additional drying cycles in air, or by heating the contrast-coated stent above a curing temperature in an oven with a controlled ambient such as vacuum, nitrogen, or air or by delivering energy to the coating via gamma or ebeam energy. Sterilization may employ, for example, gamma-ray irradiation, e-beam radiation, ethylene oxide gas, or hydrogen peroxide gas plasma sterilization techniques. The contrast drug/polymer coated stent may be packaged, shipped, and stored in a suitable package until it is used.

A delivery catheter may be coupled to the coated stent (Block 435). The delivery catheter may include an inflatable balloon that is positioned between the coated stent and the catheter and used for deploying the coated stent in the body. Alternatively, the delivery catheter may include a sheath that retracts to deploy a self-expanding version of the coated stent.

In one exemplary method, fully processed contrast-coated stents are reduced in diameter and placed into the distal end of the catheter to form an interference fit, which secures the stent onto the catheter. The catheter with the stent may be placed in a catheter package and sterilized prior to shipping and storing. Before clinical use, the stent is sterilized by any appropriate or medically conventional means.

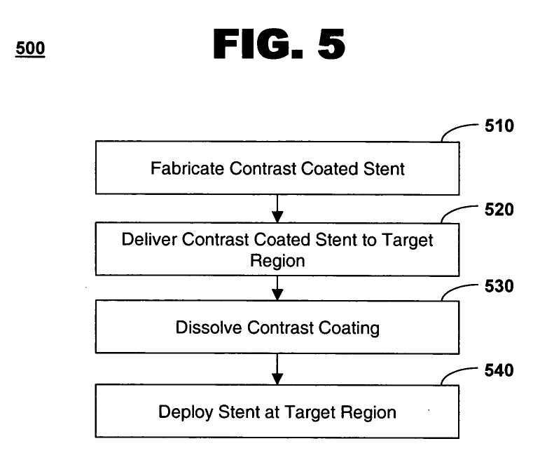

FIG. 5 shows a method of treating a vascular condition using a contrast-coated stent made in accordance with the present and referred to generally as method 500. Method 500 begins by fabricating a contrast-coated stent including at least one drug-polymer layer (Block 510). The contrast-coated stent may be fabricated using the method illustrated in FIG. 4.

When ready for deployment, the contrast-coated stent is inserted into a vessel of the body and delivered to the target region within the patient (Block 520). The stent is inserted typically in a controlled environment such as a catheter lab or hospital. The delivery catheter, which helps position the contrast coated stent in a vessel of the body, is typically inserted through a small incision of the leg and into the femoral artery, and directed through the vascular system to a desired place in the vessel. Guide wires threaded through an inner lumen of the delivery catheter assist in positioning and orienting the coated stent. The position of the coated stent may be monitored, for example, with a fluoroscopic imaging system or an x-ray viewing system. The visualization of the stent as it moves through the vessel is aided by the contrast medium coated on the stent.

The contrast coating on the stent dissolves as the stent is advanced through the patient's vessel (Block 530). As detailed above, the concentration and thickness of the contrast coating may be manipulated depending on the application. In one embodiment, the coating is of sufficient quantity such that it does not dissolve completely until the stent reaches the target region, thereby aiding the visualization of the stent as it progresses through the vessel. In another embodiment, the coating is a minimal coating such that it dissolves quickly upon insertion into the vessel, while still offering the protection to the underlying drug polymer layer as it is prepared and shipped prior to insertion.

The stent is then deployed (Block 540). The stent is deployed, for example, by expanding the stent with a balloon or by extracting a sheath that allows a self-expandable stent to enlarge after positioning the stent at a desired location within the body. At this time any remaining contrast coating will dissolve, leaving behind a drug-polymer coated stent.

Once the coated stent is deployed, the therapeutic agents in the drug-polymer coating are eluted. The elution rates of the therapeutic agents into the body and the tissue bed surrounding the stent framework are based on the polymers, thickness of the drug-polymer coating and any cap coating, and the distribution and concentration of the therapeutic agents contained therein, among other factors.

It is important to note that FIGS. 1-5 illustrate specific applications and embodiments of the present invention, and is not intended to limit the scope of the present disclosure or claims to that which is presented therein. Upon reading the specification and reviewing the drawings hereof, it will become immediately obvious to those skilled in the art that myriad other embodiments of the present invention are possible, and that such embodiments are contemplated and fall within the scope of the presently claimed invention.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. A system for treating a vascular condition, the system comprising:
a catheter;
a stent disposed on the catheter, the stent including a stent framework,
a drug coating disposed on the stent framework, and
a contrast medium substantially covering at least an outer surface of the drug coating disposed on an outer surface of the stent framework.

2. The system of claim 1 wherein the contrast medium is coated on the framework to a thickness which allows the stent to be delivered to a target region of a vessel before fully dissolving.

3. The system of claim 1 wherein the contrast medium substantially covers an inner surface of the stent framework.

4. The system of claim 3 wherein the contrast medium substantially covers an outer surface of the drug coating disposed on an inner surface of the stent framework.

5. The system of claim 1 wherein the catheter includes a balloon to expand the stent.

6. The system of claim 1 wherein the catheter includes a sheath that retracts to allow expansion of the stent.

7. The system of claim 1 wherein the stent framework comprises one of a metallic base or a polymeric base.

8. The system of claim 1 wherein the stent framework comprises a material selected from the group consisting of stainless steel, nitinol, tantalum, MP35N alloy, platinum, titanium, a chromium-based alloy, a suitable biocompatible alloy, a suitable biocompatible material, a biocompatible polymer, and a combination thereof.

9. A method of treating a vascular condition, the method comprising:
delivering a drug coated stent with contrast coating to a target region of a vessel via a catheter;
dissolving the coating while the stent is delivered to the target region; and
deploying the stent at the target region.

10. The method of claim 9 wherein the contrast coating has a thickness to allow an outer surface of the drug coated stent to be substantially covered during delivery of the stent to the target region.

11. A method of protecting a drug coated stent, the method comprising:
applying a contrast medium solution to at least an outer surface area of a drug coated stent; and
drying the applied contrast medium solution to form a contrast coating.

12. The method of claim 11 wherein applying the contrast medium solution comprises one of dipping, brushing, painting, spraying, or dispensing.

13. The method of claim 11 wherein the contrast medium solution is comprised of a contrast medium and a dilutant.

14. The method of claim 11 wherein applying a contrast medium solution to at least an outer surface area of a drug coated stent comprises applying the contrast medium solution to the drug-coated stent disposed on a balloon catheter.
